(19) United States
(12) Patent Application Publication
(10) Pub. No.: US 2025/0177438 A1
(43) Pub. Date: Jun. 5, 2025

(54) FREEZE DRIED, READY TO CONSUME NUTRICOSMETIC PRODUCTS AND PROCESSES FOR THEIR PREPARATION

(71) Applicant: VITALMELT LTD., Petah Tikva (IL)

(72) Inventor: David KLIGER, Ramat Gan (IL)

(21) Appl. No.: 18/842,039

(22) PCT Filed: Mar. 5, 2023
(86) PCT No.: PCT/IL2023/050223

§ 371 Date: Aug. 27, 2024

Related U.S. Application Data
(60) Provisional application No. 63/317,038, filed on Mar. 6, 2022.

(51) Int. Cl. A61K 33/06; A23L 19/00; A61K 8/19; A61K 8/27; A61K 8/65; A61K 8/67; A61K 8/92; A61K 9/00; A61K 9/19; A61K 31/07; A61K 31/355; A61K 31/375; A61K 31/4415; A61K 31/51; A61K 31/519; A61K 33/26; A61K 33/30; A61K 33/34; A61K 38/39; A61K 47/44; A61K 47/46; A61Q 19/00

(52) U.S. Cl. CPC A61K 33/06; A23L 19/09; A61K 8/19; A61K 8/27; A61K 8/65; A61K 8/671; A61K 8/673; A61K 8/676; A61K 8/678; A61K 8/922; A61K 9/0056; A61K 9/19; A61K 31/07; A61K 31/355; A61K 31/375; A61K 31/4415; A61K 31/51; A61K 31/519; A61K 33/26; A61K 33/30; A61K 33/34; A61K 38/39; A61K 47/44; A61K 47/46; A61Q 19/00; A61K 2800/10; A61K 2800/84; A61K 2800/92

(57) ABSTRACT

Disclosed are freeze-dried nutricosmetic compositions including a mixture of at least 10% w/w of a fruit and/or vegetable puree; and at least 0.5% w/w of an exogenous nutricosmetic; wherein the nutricosmetic composition is freeze dried to obtain a ready to be consumed product form which melts in the mouth.

### TECHNOLOGICAL FIELD

The present invention provides a freeze-dried solid nutricosmetic composition in a product form that is capable of instantly melting in the mouth, processes for its preparation and use of same as nutricosmetics and nutri-beauty products.

### BACKGROUND

Nutraceuticals are a group of products that are more than food but less than pharmaceuticals. In most countries, nutraceuticals are taken as part of dietary supplements. Frequency of nutraceuticals use is 50%-70% in developed countries' population and this number is increasing by the age and at times may be prescribed due to nutritional deficiencies. Nutricosmetics can also affect cosmetic and appearance.

For example, skin, hair, and nails can be indicative of overall health. The appearance of nails, skin and hair can indicate the body condition and are influenced, inter alia, by nutrition. Diet constraint, imbalanced diet and eating disorders can cause nutritional deficiencies, rendering such deficiencies as acute health, cosmetic and beauty problem.

Common nutricosmetics influencing hair, nails and skin appearance and well-being include Zinc, Copper, Antioxidants, Collagen, cysteine, methionine, biotin, Vitamin C, folic acid, vitamin b complex, hyaluronic acid, Vitamin A, Vitamin D, Vitamin E, Pantothenic Acid, Iodine, Inositol, Niacin, Choline, Caffeine, GLA (Gamma Linolenic Acid), Theanine, Siberian ginseng, Pomegranate extract, Astaxanthin, Grape seed extract, Cannabinoids (such as THC, CBD and Terpenes), plants terpenes.

Accordingly, there is a need in the art for improved nutricosmetic compositions, which can be orally consumed, in order to efficiently and cost effectively affect skin, nails and/or hair appearance and health by providing nutricosmetics is a safe and robust manner.

### SUMMARY

The present invention provides a nutricosmetic product/composition including at least 10% w/w of a fruit puree and/or vegetable puree; and at least 5% w/w of an exogenously added nutricosmetic; wherein the nutricosmetic composition is freeze dried into a ready to be consumed product form, which melts in the mouth. According to some embodiments, the nutricosmetic product may further include at least 5% w/w of an edible oil.

Advantageously, the components of the nutricosmetic product maintain at least about 75% and even at least about 90% of their initial nutritional value.

As a further advantage, the nutricosmetic product is devoid of un-natural preservatives and all ingredients are at least 95% natural.

According to some embodiments, advantageously, the ready to consume nutricosmetic product has a good taste which encourage compliance.

According to some embodiments, due to the product being capable of melting in the mouth, the herein disclosed compositions are particularly useful for administration to a variety of subjects, such as, for example, subjects in need to nutricosmetics.

According to some embodiments, there are provided herein advantageous nutricosmetic compositions, which are ready for oral consumption, cost efficient and effective in providing various cosmetic remedies to subjects in need thereof, by preventing and/or improving health and/or appearance of the subjects. In some embodiments, the compositions disclosed herein are advantageous, as they exhibit improved bioavailability of nutricosmetic substances included within the compositions, thereby facilitating an enhanced effect thereof. In some embodiments, the enhanced effect may be, for example, on health and/or appearance of skin, nails and/or hair of the subject.

According to some embodiments, there is provided a freeze-dried nutricosmetic composition comprising a mixture of:
(a) at least 10% w/w of a fruit and/or vegetable puree; and
(b) at least 0.5% w/w of an exogenous nutricosmetic;
wherein the nutricosmetic composition is freeze dried to obtain a ready to be consumed product form which melts in the mouth.

According to some embodiments, the composition may further include at least 5% w/w of an edible oil.

According to some embodiments, the composition includes less than about 5% w/w water.

According to some embodiments, the composition includes about 30%-70% w/w of the fruit and/or vegetable puree.

According to some embodiments, the composition includes about 10%-40% w/w of the edible oil.

According to some embodiments, the composition includes about 1%-50% w/w of the nutricosmetic.

According to some embodiments, includes the nutricosmetic may include one or more of a vitamin, a mineral, a herb, a herb extract, a herb derivative, medicinal mushrooms, medicinal mushrooms extract, Cannabis extract, Collagen, glycosaminoglycan, hyaluronic acid (HA), caffeine, probiotics, probiotics bacterium, dietary fiber, dietary supplements, Zinc, Copper, antioxidants, cysteine, methionine, biotin, Astaxanthin, Grape seed extract, Vitamin C, folic acid, vitamin B complex, Vitamin A, Vitamin D, Vitamin E, Pantothenic Acid, Iodine, Inositol, Niacin, Choline, coconut extract, coconut oil, or any combination thereof. For example, a combination of Vitamin A and hyaluronic acid.

According to some embodiments, the nutricosmetic includes Collagen.

According to some embodiments, the nutricosmetic includes Hyaluronic acid (HA).

According to some embodiments, the nutricosmetic may be encapsulated.

According to some embodiments, the composition may further include a flavoring agent.

According to some embodiments, the composition may further include a sweetener.

According to some embodiments, the composition may further include at least about 15% w/w of the sweetener.

According to some embodiments, the composition may include about 20%-40% w/w of the sweetener.

According to some embodiments, the edible oil is an oil having a melting temperature below about 50° C. According to some embodiments, the edible oil is an oil having a melting temperature below about 40° C. According to some embodiments, the edible oil may be selected from a vegetable oil, a saturated fatty acid, unsaturated fatty acid or any combinations thereof. In some embodiments, the edible oil is coconut oil.

According to some embodiments, the composition may include about 1%-5% w/w of water.

According to some embodiments, there is provided a process for the preparation of a freeze-dried nutricosmetic composition, the process includes the steps of:
a. mixing a nutricosmetic with an edible oil to obtain a first mixture, wherein the mixing is performed at a temperature of below 50° C.;
b. mixing the first mixture with a fruit and/or vegetable puree to obtain a second mixture;
c. freeze drying said second mixture until water content is less than about 5% w/w of the product; thereby producing a freeze-dried nutricosmetic composition having a ready to be consumed product form, which melts in the mouth.

According to some embodiments, step (a) includes mixing at least 5% w/w of the final product of the nutricosmetic with at least % w/w of the final product of the edible oil. In some embodiments, step (b) includes mixing the first mixture with at least at least 10% w/w of the final product of the fruit and/or vegetable puree.

According to some embodiments, the process may further include adding a sweetener at step (b).

In some embodiments, at least 15% w/w of the final product of sweetener is added.

According to some embodiments, the second mixture is cast into a mold prior to the freeze drying. According to some embodiments, the second mixture is frozen and shaped prior to freeze drying.

According to some embodiments, there is provided a method for administering a nutricosmetic to a subject in need thereof, the method includes inserting into the subject's mouth the nutricosmetic composition disclosed herein.

According to some embodiments, the method is for providing enhanced cosmetic effect to the subject.

According to some embodiments, the enhanced cosmetic effect includes an effect on skin, nails and/or hair of the subject.

According to some embodiments, the nutricosmetic compositions disclosed herein are for use in administration of a nutricosmetic to a subject in need thereof.

According to some embodiments, the subject is a subject undergoing or having undergone a cosmetic treatment.

Certain embodiments of the present disclosure may include some, all, or none of the above advantages. One or more technical advantages may be readily apparent to those skilled in the art from the descriptions and claims included herein. Moreover, while specific advantages have been enumerated above, various embodiments may include all, some or none of the enumerated advantages.

### DETAILED DESCRIPTION OF EMBODIMENTS

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

According to some embodiments, there is provided a nutricosmetic product/composition comprising: at least 10% w/w of a fruit puree; and at least 5% w/w of an exogenously added nutricosmetic; wherein the composition is freeze dried into a ready to be consumed product form, which melts in the mouth. In some embodiments, the composition may further include at least 5% w/w of an edible oil.

According to some embodiments, there is provided a nutricosmetic product/composition comprising: at least 10% w/w of a fruit puree; and at least 5% w/w of an exogenously added nutricosmetic; wherein the composition is freeze dried into a ready to be consumed product form, which melts in the mouth. According to some embodiments, the nutricosmetic product may further include at least 5% w/w of an edible oil.

As used herein the terms “nutraceutical”, “nutricosmetic”, “nutria-cosmetic”, “beauty from within”, “beautyceutical” and “bioceutical” may be used interchangeably and refer to any substance that is a food, food-derived, or part of a food and provides cosmetical, medical or health benefits, including, for example, the prevention and/or improvement of various cosmetic/appearance/health related conditions of skin, hair and/or nails, Glowing of hair, skin or nails. In some embodiments, the nutricosmetic compositions disclosed herein may provide beneficial effect on, for example, but not limited to: wrinkles, age spots, brown spots, freckles, sagging skin, folds, volume loss, skin tone, skin texture, acne, acne scaring, nail appearance, nails health, nails composition, hair health (for example, length, strength, abundancy, etc.), and the like, or any combinations thereof).

According to some embodiments, the nutricosmetic may be a dietary supplement.

According to some embodiments, the nutricosmetic may be or may include one or more vitamins, minerals, fatty acids, herbs or other botanicals, amino acids, and substances such as enzymes, organ tissues, glandulars, and metabolites. As a non-limiting example, the nutricosmetic may be a vitamin, such as but not limited to a multivitamin (which includes a combination/mixture of vitamins and/or various minerals). As a non-limiting example, the nutricosmetic may include, for example, such substances as, but not limited to: Medicinal mushrooms, Medicinal mushrooms extract, Cannabis extracts, Collagen, Glycosaminoglycans, hyaluronic acid (HA), caffeine, probiotics, probiotics bacterium, Dietary fiber, dietary supplements, Zinc, Copper, Antioxidants, cysteine, methionine, biotin, Vitamin C, folic acid, Vitamin B(s), vitamin B complex, Vitamin A, Vitamin D, Vitamin E, Vitamin K, Zinc, Mg, Calcium, Copper, Selenium, Iron, Pantothenic Acid, Iodine, Inositol, Niacin, Choline, Astaxanthin, Grape seed extract, Coconut extract, coconut oil, fatty acids and the like, or any combination thereof. Each possibility is a separate embodiment. For example, a nutricosmetic may include Vitamin A and hyaluronic acid.

According to some embodiments, the nutricosmetic may be an extract or concentrate. As a non-limiting example, the nutricosmetic may be a herb extract, such as cannabis extract. According to some embodiments, the nutricosmetic may be compound extracted from an herb such as a cannabinoid. According to some embodiments, the cannabinoid may be THC or CBD.

According to some embodiments, the composition may include about 0.0005% to about 40% w/w of the exogenous nutricosmetic. According to some embodiments, the composition may include about 0.01% to about 38% w/w of the nutricosmetic. According to some embodiments, the composition may include about 0.05% to about 35% w/w of the nutricosmetic. According to some embodiments, the composition may include about 0.5% to about 35% w/w of the nutricosmetic. According to some embodiments, the composition may include about 1% to about 30% w/w of the nutricosmetic. According to some embodiments, the composition may include about 5% to about 30% w/w of the nutricosmetic. According to some embodiments, the composition may include about 12% to about 25% w/w of the of the nutricosmetic. According to some embodiments, the composition may include about 0.005% to about 8% w/w, or about 0.05% to about 7%, or about 0.5% to about 5% of the exogenous nutricosmetic. According to some embodiments, the composition may include about 0.2% to about 2% w/w, or about 0.5% to about 1% of the of the exogenous nutricosmetic. According to some embodiments, the composition may include at least 0.05%, at least 0.5%, at least 1%, at least 2%, at least 5%, at least 10%, or at least 15% w/w of the nutricosmetic. According to some embodiments, the composition may include about 10%, about 15%, about 20%, about 25%, about 30% or about 40% w/w of the of the exogenous nutricosmetic. Each possibility is a separate embodiment.

According to some embodiments, the nutricosmetic may be encapsulated. As used herein the term “encapsulated” relates to a physical-chemical technique or process by which an ingredient may be surrounded by a coating e.g., a fat, liposome or polymer, etc., thereby separating the ingredient from its environment. According to some embodiments, an encapsulation coating may protect an ingredient against diverse conditions including moisture, temperature fluctuations etc. According to some embodiments, an encapsulation coating may serve as a carrier system for an active ingredient, such as a nutricosmetic.

According to some embodiments, the amount of the exogenous nutricosmetic in the composition depends on the type of the nutricosmetic.

According to some embodiments, the composition may include about 2% to about 10% w/w of the nutricosmetic. According to some embodiments, the composition includes about 12% to about 25% w/w of the of the nutricosmetic. According to some embodiments, the composition includes about 25% to about 50% w/w of the of the nutricosmetic. According to some embodiments, the composition includes at least 10%, or at least 15% w/w of the nutricosmetic. According to some embodiments, the composition includes about 10%, about 15%, about 20%, about 25%, about 30%, about 40% or about 50% w/w of the of the nutricosmetic. Each possibility is a separate embodiment.

According to some embodiments, the nutricosmetic may be in the form of a powder, a microcapsule, a liquid, a softgel, or the like. Each possibility is a separate embodiment.

As used herein, the term “exogenously added” with regards to the nutricosmetic refers to the nutricosmetic as a separate component and not as a component of other ingredients of the composition. As a non-limiting example, the nutricosmetic may be vitamin C in which case it refers to vitamin C added as a separate component different from the vitamin C of the fruit puree.

According to some embodiments, the term “fruit puree” may refer to any type of fruit or combination of fruits that has been cooked, ground, pressed, blended or sieved until it reaches a consistency of a creamy paste or liquid.

According to some embodiments, the composition may include about 15% to about 50% w/w of the fruit puree. According to some embodiments, the composition includes about 20% to about 40% w/w of the fruit puree. According to some embodiments, the composition includes at least 15%, or at least 20% w/w of the fruit puree. According to some embodiments, the composition includes about 20%, about 25%, about 28%, about 30%, about 35% or about 40% w/w of the fruit puree. Each possibility is a separate embodiment.

According to some embodiments, the composition may, in addition to, or as an alternative to the fruit puree, include a vegetable puree.

Thus in some embodiments, the composition may include a fruit puree and/or a vegetable puree.

According to some embodiments, the term “vegetable puree” may refer to any type of vegetable or combination of vegetables that has been cooked, ground, pressed, blended or sieved until it reaches a consistency of a creamy paste or liquid.

According to some embodiments, the composition may include about 15% to about 70% w/w of the vegetable puree. According to some embodiments, the composition may include about 20% to about 60% w/w of the vegetable puree. According to some embodiments, the composition may include at least 15%, or at least 20% w/w of the vegetable puree. According to some embodiments, the composition may include about 20%, about 25%, about 28%, about 30%, about 35%, about 40%, w/w about 50% w/w or about 55% w/w of the vegetable puree. Each possibility is a separate embodiment.

According to some embodiments, the amount of vegetable puree in the composition depends on the type of the exogenous nutricosmetic.

According to some embodiments, the composition is suitable for use as a food product. The term “food product” as used herein should be understood to encompass a product that is orally consumed to provide at least one nutricosmetic to a subject. The food product is in a solid form that is ready to be consumed by directly introducing to the mouth.

It is further defined that the composition of the present invention is “freeze-dried”, also known as “lyophilization” and “cryodesiccation”, resulting in a chewable and/or meltable solid form. As used herein, “freeze drying” refers to a low temperature dehydration process that involves freezing the product, lowering pressure, followed by a removing of ice by sublimation. This is in contrast to dehydration by most conventional methods that evaporate water using heat.

Additionally, the composition of the present invention is defined to be a “ready to be consumed product”, thus it does not have to be further prepared in any manner. It does not have to be further cooked, rehydrated, steamed, mixed with a liquid (hot or cold), fried, microwaved or any other form of preparation for being consumed by a subject.

As used herein, the term “edible oil” is fat of plant, animal or microbial origin. According to some embodiments, the edible oil is meltable at temperatures below 40° C., below 37° C., below 35° C., below 30° C. or at room temperature and is suitable for food use. Non-limiting examples of suitable oils include coconut oil, palm oil and palm kernel oil, soybean oil, canola oil (rapeseed oil), corn oil, peanut oil, sunflower oil, olive oil and other vegetable oils. Each possibility is a separate embodiment. According to some embodiments, the edible oil is coconut oil.

According to some embodiments, the composition may include about 0.1% to about 10% of the edible oil. According to some embodiments, the composition may include about 10% to about 40% w/w of the edible oil. According to some embodiments, the composition includes about 15% to about 30% w/w of the edible oil. According to some embodiments, the composition includes at least 15%, or at least 20% w/w of the edible oil. According to some embodiments, the composition includes about 0.1%, about 1%, about 2%, about 3%, about 4%, about 5%, about 10%, about 15%, about 20%, about 25%, about 30% or about 40% w/w of the edible oil. Each possibility is a separate embodiment. Optionally, the composition may not include an edible oil.

According to some embodiments, the composition has less than about 5% w/w water. The term “less than 5% w/w of water” should be understood to relate to the amount of water present in the final food product of the invention. Since the food product of the invention is solid and chewable/meltable, the amount of hydration in the product to be consumed is reduced to less than 5% by weight of the product.

According to some embodiments, the composition has between about 1% to about 5% of water. In some embodiments, the composition has about 1%, 2%, 3%, 4%, 5% w/w of the product of water. Each possibility is a separate embodiment.

According to some embodiments, the composition may further include a sweetener. According to some embodiments, the sweetener may be a natural sweetener. Non-limiting examples of suitable natural sweeteners include sugar, glucose, fructose, stevia, erythritol, Xylitol, Mannitol, Yacon syrup, honey, maple, molasses, coconut sugar, Monk fruit sweetener and any combination thereof. Each possibility is a separate embodiment. According to some embodiments, the sweetener may be a synthetic sweetener. Non-limiting examples of suitable natural sweeteners include aspartame, Acesulfame potassium, Advantame, Aspartame-acesulfame salt, Cyclamate, Neotame, Neohesperidin, Sacchari, Sucralose or any combination thereof. Each possibility is a separate embodiment.

According to some embodiments, the sweetener is white sugar.

According to some embodiments, the composition comprises at least about 10%, at least about 15%, at least about 20% or at least about 25% or at least about 30% w/w of the white sugar or other sweetener. Each possibility is a separate embodiment. According to some embodiments, the composition comprises about 10% to about 40% w/w of white sugar or other sweetener. According to some embodiments, the composition comprises about 15% to about 30% w/w of white sugar or other sweetener. According to some embodiments, the composition comprises about 10%, about 15%, about 20%, about 25% or about 30% w/w of white sugar or other sweetener. Each possibility is a separate embodiment.

In some embodiments, the nutricosmetic food product of the invention may further include further include in addition to, or alternatively to the sweetener, at least one flavoring agent. It should be noted that said flavoring agent relates to any type of edible substance added to a product of the invention (at any step of product preparation) that adds and/or alters and/or intensifies the flavor of a product of the invention through the sense of taste and/or smell. In some embodiments said at least one flavoring agent is a natural flavoring substance (i.e. flavoring substances obtained from plant or animal raw materials, by physical, microbiological or enzymatic processes), a nature-identical flavoring substance (i.e. flavoring substances that are obtained by synthesis or isolated through chemical processes, which are chemically and organoleptically identical to flavoring substances naturally present in products intended for human consumption) or an artificial flavoring substance (i.e. chemically produced flavoring substances that are typically chemically different from the corresponding natural flavoring substance however, in sensory characteristics are the same as natural ones), and any combination thereof.

In some embodiments, said at least one flavoring agent is selected from the group consisting of a sweetening agent (such as a sweetening saccharide, sugar, sugar substitutes), fructose, galactose, flavoring extracts (such as spices and herb flavors, including vanilla, cinnamon, nutmeg, cardamom and so forth; fruit flavors including banana, strawberry, berry, grape, orange, pineapple, lemon and so forth) and any combinations thereof.

According to some embodiments, the flavoring agent may be lemon salt.

According to some embodiments, the composition comprises at least about 0.5%, at least about 1%, at least about 1.5% or at least about 2% w/w of lemon salt or other flavoring agent. Each possibility is a separate embodiment. According to some embodiments, the composition comprises about 0.5% to about 5% w/w of lemon salt or other flavoring agent. According to some embodiments, the composition comprises about 1% to about 3% w/w of lemon salt or other flavoring agent. According to some embodiments, the composition comprises about 0.5%, about 1%, about 1.5%, about 2% or about 2.5% w/w of lemon salt or other flavoring agent. Each possibility is a separate embodiment.

According to some embodiments, the composition may further include a thickening component. In some embodiments, the at least one thickening component may be a monosaccharide, oligosaccharide (for example disaccharides, like sugar), polysaccharide (for example starch, which can be either native, none-native, modified or processed starch and any combinations thereof) or any combinations thereof. In other embodiments, at least one thickening component is starch. In yet further embodiments, the at least one thickening component is a mixture of starch and sugar. In some embodiments, the at least one thickening component is starch selected from potato starch, tapioca starch, rice starch, corn starch, wheat starch, maze starch, cassava starch, sweet potato starch or any combinations thereof.

In some embodiments, at least one thickening component is between about 10% to about 70% weight of the product. In some embodiments, at least one thickening component is about 10%, 20%, 25%, 30%, 35%, 40%, 45%, 50%, 55%, 60%, 65%, 70% weight of the product. Each possibility is a separate embodiment.

In some embodiments, a nutricosmetic product of the invention has a mechanical compressibility of at least 5 N/cm²/or Pa^{“1}. In some embodiments, a food product of the invention has a mechanical compressibility in the range of between 5 N/cm² and 20 N/cm².

In some embodiments, a food product of the invention has a density of at least 120 mg/cm³. In some embodiments, a food product of the invention has a density in the range of between 120 mg/cm³ and 500 mg/cm³.

In some embodiments, the composition is a non-dairy food product, thus it does not contain any dairy component. Alternatively, the composition may further include at least one dairy component.

According to some embodiments, the composition further includes a natural preservative, an oxidating agent, an emulsifier or other food grade agent optimizing the structure, chewability, meltability, preservation or other feature/characteristic of the composition.

According to some embodiments, there is further provided a process for the preparation of a freeze-dried nutricosmetic composition, the process comprising the steps of:
a. mixing a nutricosmetic with an edible oil to obtain a first mixture, wherein the mixing is performed at a temperature of below about 50° C.;
b. mixing the first mixture with a fruit puree and/or vegetable puree to obtain a second mixture;
c. freeze drying the second mixture until water content is less than about 5% w/w of the product; thereby producing a freeze-dried nutricosmetic composition having a ready to be consumed product form, which melts in the mouth.

According to some embodiments, step (a) comprises mixing at least 5% w/w nutricosmetic with at least 10% w/w of the edible oil, wherein the weight percent relates to the to the total weight of the final product.

According to some embodiments, step (b) comprises mixing the first mixture with at least at least 10% w/w of the fruit and/or vegetable puree.

According to some embodiments, the process further includes adding a sweetener at step (a) or (b). According to some embodiments, at least 15% w/w of sweetener is added, wherein the weight percent relates to the to the total weight of the final product.

According to some embodiments, the second mixture is cast into a mold prior to the freeze drying.

According to some embodiments, the second mixture is frozen and shaped prior to freeze drying.

According to some embodiments, there is further provided a process for the preparation of a freeze-dried nutricosmetic composition, the process comprising the steps of: mixing a nutricosmetic with a fruit and/or vegetable puree to obtain a mixture and freeze drying the mixture until water content is less than about 5% w/w of the product; thereby producing a freeze-dried nutricosmetic composition having a ready to be consumed product form, which melts in the mouth.

According to some embodiments, there is provided a method for administering a nutricosmetic to a subject in need thereof, the method comprising inserting into the subject's mouth, the herein disclosed nutricosmetic composition.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Example 1: Process for the Preparation of the Herein Disclosed Nutricosmetic Composition Comprising Multivitamin and/or Collagen

The ingredients of the multivitamin composition are set forth in Table 1 below.

TABLE 1

| | | Percent |
|---|---|---|
| Raw Materials | Weight | Weight |
| Fruit Puree | 30.4 | 30% |
| White sugar | 21.6 | 22% |
| Coconut oil | 13 | 13% |
| Multivitamin | 35 | 35% |
| Total 1 Pack | 100 gm | 100% |

The ingredients of the Collagen composition are set forth in Table 2 below.

TABLE 2

| | | Percent |
|---|---|---|
| Raw Materials | Weight | Weight |
| Fruit Puree | 30.4 | 30% |
| White sugar | 21.6 | 22% |
| Coconut oil | 13 | 13% |
| Collagen | 35 | 35% |
| Total 1 Pack | 100 gm | 100% |

Initially, the multivitamin or Collagen was mixed into the coconut oil until fully dissolved. The multivitamin—coconut oil mixture was then mixed with the remaining ingredients to a mash-like mixture. The mash-like mixture was poured into IQF machine (individual quick freezing), moulds or by depository system, frozen, and subsequently lyophilized until the total amount of water was below 3% so as to obtain an essentially solid, yet meltable single-dose composition.

### Example 2: Evaluation of the Nutritional Value of the Herein Disclosed Nutricosmetic Composition Comprising Multivitamin

The components value of a freeze-dried single dose composition including a mixture of multivitamin was verified by suitable analysis methods. The amount (weight pre dose) of the listed components was determined prior to mixing with other ingredients, and in the final product (after freeze drying). The results are presented in Table 3.

TABLE 3

| | | |
|---|---|---|
| Exogenous | Weight per dose in | Weight per dose in the |
| Nutricosmetic | the original premix | freeze dried final product |

| | | | | |
|---|---|---|---|---|
| Calcium | 592 | mg | 593 | mg |
| Copper | 1.73 | mg | 2.16 | mg |
| Iron | 30.1 | mg | 32.6 | mg |
| Zinc | 30.4 | mg | 38 | mg |
| Vitamin B6 | 17.2 | mg | 16.8 | mg |
| Folic Acid | 0.585 | mg | 0.675 | mg |
| Vitamin B1 | 2.37 | mg | 3.47 | mg |
| Vitamin C | 134 | mg | 181 | mg |
| Vitamin A | 2.7 | mg | 3.1 | mg |
| Vitamin E | 296 | mg | 310 | mg |

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1.-30. (canceled)

31. A freeze-dried nutricosmetic composition comprising a mixture of:
(a) at least 10% w/w of a fruit and/or vegetable puree; and
(b) at least 0.5% w/w of an exogenous nutricosmetic;
wherein the nutricosmetic composition is freeze dried to obtain a ready to be consumed product form which melts in the mouth.

32. The composition of claim 31, further comprising at least 5% w/w of an edible oil.

33. The composition of claim 31, comprising less than about 5% w/w water.

34. The composition according to claim 31, comprising about 30%-70% w/w of the fruit and/or vegetable puree.

35. The composition according to claim 32, comprising 10%-40% w/w of the edible oil.

36. The composition according to claim 31, comprising about 1%-50% w/w of the nutricosmetic.

37. The composition according to claim 31, wherein the nutricosmetic comprises: a vitamin, a mineral, a herb, a herb extract, a herb derivative, medicinal mushrooms, medicinal mushrooms extract, Cannabis extract, Collagen, glycosaminoglycan, hyaluronic acid (HA), caffeine, probiotics, probiotics bacterium, dietary fiber, dietary supplements, Zinc, Copper, antioxidants, cysteine, methionine, biotin, Vitamin C, folic acid, vitamin B complex, Vitamin A, Vitamin D, Vitamin E, Pantothenic Acid, Iodine, Inositol, Niacin, Choline, Astaxanthin, Grape seed extract, coconut extract, coconut oil, or any combination thereof.

38. The composition according to claim 31, wherein the nutricosmetic comprises Collagen and/or Hyaluronic acid (HA).

39. The composition according to claim 31, further comprising a flavoring agent and/or a sweetener.

40. The composition according to claim 39, comprising at least 15% w/w of the sweetener.

41. The composition according to claim 40, comprising 20%-40% w/w of the sweetener.

42. The composition according to claim 40, wherein the edible oil is an oil having a melting temperature below about 50° C.

43. The composition according to claim 31, wherein the edible oil is selected from a vegetable oil, a saturated fatty acid, unsaturated fatty acid or any combinations thereof.

44. The composition of claim 43, wherein the edible oil is coconut oil.

45. A process for the preparation of a freeze-dried nutricosmetic composition, the process comprising the steps of:
a. mixing a nutricosmetic with an edible oil to obtain a first mixture, wherein the mixing is performed at a temperature of below 50° C.;
b. mixing the first mixture with a fruit and/or vegetable puree to obtain a second mixture;
c. freeze drying said second mixture until water content is less than about 5% w/w of the product; thereby producing a freeze-dried nutricosmetic composition having a ready to be consumed product form, which melts in the mouth.

46. The process of claim 45, wherein step (a) comprises mixing at least 5% w/w of the final product of the nutricosmetic with at least % w/w of the final product of the edible oil.

47. The process of claim 45, wherein step (b) comprises mixing the first mixture with at least at least 10% w/w of the final product of the fruit and/or vegetable puree.

48. The process of claim 45, further comprising adding sweetener at step (b).

49. The process of claim 45, wherein the second mixture is cast into a mold prior to the freeze drying.

50. The process of claim 45, wherein the second mixture is frozen and shaped prior to freeze drying.
